(19) United States
(12) Patent Application Publication
(10) Pub. No.: US 2019/0392947 A1
(43) Pub. Date: Dec. 26, 2019

(54) Fitting System And Medical Accessory Carrier

(71) Applicant: FRESENIUS MEDICAL CARE DEUTSCHLAND GMBH, Bad Homburg (DE)

(72) Inventors: Olaf SCHERMEIER, Frankfurt (DE); Kirill KOULECHOV, Bad Vilbel (DE)

(73) Assignee: FRESENIUS MEDICAL CARE DEUTSCHLAND GMBH, Bad Homburg (DE)

(21) Appl. No.: 16/480,684

(22) PCT Filed: Jan. 29, 2018
(86) PCT No.: PCT/EP2018/052121

§ 371 Date: Jul. 25, 2019

(30) Foreign Application Priority Data
Jan. 30, 2017 (DE) 10 2017 201 436.9

(51) Int. Cl. G16H 40/20; G06Q 10/08

(52) U.S. Cl. CPC G16H 40/20; G06Q 10/087

(57) ABSTRACT

The invention relates to a pick-and-place system for the equipping of medical accessory holders, in particular medical accessory boxes, for a patient treatment. The pick-and-place system comprises a storage apparatus for a plurality of medical accessories of at least two types, a separating apparatus for medical accessories and an assembling area for a medical accessory holder. The pick-and-place system furthermore comprises an accessory database for data sets on the plurality of medical accessories and in respect of one or more patient treatments as well as a data storage apparatus for the accessory database designed to store the accessory database. The pick-and-place system furthermore comprises one or more data interfaces, wherein at least one of the data interfaces is designed to receive one or more selection parameters, in particular treatment data or patient data, relative to the treatment of the patient. Lastly, the pick-and-place system comprises a data processing apparatus configured to perform a selection procedure. Said selection procedure comprises at least one of the following procedural steps: determining in one procedural step one or more accessory set parameters characterizing a medical accessory set from one or more medical accessories applicable to the treatment of the patient by means of the accessory database and at least on the basis of the selection parameter; actuating the separating apparatus for medical accessories at least on the basis of the accessory set parameter in a further procedural step. The separating apparatus for medical accessories is thereby configured to extract the medical accessories of the accessory set from the storage apparatus and deliver same to the assembling area pursuant to the activation.

The present invention relates to the field of medical technology and hospital logistics and in particular to a pick-and-place system for the equipping of medical accessory holders as well as a medical accessory box for such a pick-and-place system.

In a treatment of a patient, in particular a medical treatment, in addition to the actual treatment itself, it is generally necessary to provide medical accessories for the respective treatment. Such a treatment is in particular a dialysis treatment of a patient in which the respective dialysis treatment to be performed depends on the respective patient and, depending in turn thereon, requires different medical accessories. On the one hand, medical advances have yielded more varied treatments with the accessories thereto required becoming more numerous, diversified and specialized, whereby this can result in increasing expenditure in providing medical accessories for the treatment of a patient. On the other hand, there is also the increasing desire in the medical sector, in particular in medical technology and hospital logistics which lead to the actual medical treatments, for an optimization and efficient use of resources while maintaining treatment quality at the same or even a higher level. In particular with recurring treatments, as is especially the case with dialysis treatments, there is thereby the need to adapt the treatment to the respective patients and/or make efficient use of the available resources.

The invention is based on the task of providing medical accessories, in particular as regards the logistics, improving the selecting of suitable medical accessories and/or the efficient use of medical accessories, increasing treatment safety and/or quality and/or reducing the workload on individuals involved in a patient treatment.

The invention respectively solves this task by a pick-and-place system for equipping medical accessory holders in accordance with the teaching of independent claim 1 and a medical accessory holder in accordance with the teaching of independent claim 15. Preferential embodiments, further developments or variants in particular constitute the subject matter of the independent claims. The subject matter of the claims is expressly made a part of the specification disclosure.

A first aspect of the invention relates to a pick-and-place system for equipping medical accessory holders, in particular medical accessory boxes, for a patient treatment. The pick-and-place system comprises a storage apparatus for a plurality of medical accessories of at least two types, a separating apparatus for medical accessories, and an assembling area for a medical accessory holder. The pick-and-place system additionally comprises an accessory database for data sets on the plurality of medical accessories and in respect of one or more patient treatments as well as a data storage apparatus for the accessory database designed to store the accessory database. The pick-and-place system furthermore comprises one or more data interfaces, whereby at least one of the data interfaces is designed to receive one or more selection parameters, in particular treatment data or patient data, relative to the treatment of the patient. Lastly, the pick-and-place system comprises a data processing apparatus configured to perform a selection procedure. This selection procedure comprises at least the following procedural steps: Determining in one procedural step one or more accessory set parameters characterizing a medical accessory set from one or more medical accessories applicable to the treatment of the patient by means of the accessory database and at least on the basis of the selection parameter; actuating the separating apparatus for medical accessories in a further procedural step at least on the basis of the accessory set parameter. The separating apparatus for medical accessories is thereby configured to extract the medical accessories of the accessory set from the storage apparatus for medical accessories and deliver same to the assembling area for the medical accessory holder pursuant to activation.

Broadly defined, the accessory holder, the plurality of medical accessories, the data sets on the plurality of medical accessories and/or the accessory set, in particular the accessory holder equipped with the medical accessory set, are not component parts of the pick-and-place system according to the invention. Preferably, however, in a narrower interpretation, one or more medical accessory holders, the plurality of medical accessories, the data sets on the plurality of medical accessories and/or the accessory set, in particular the accessory holder equipped with the accessory set, can be a component part of the pick-and-place system according to the invention or at least a preferential embodiment of the pick-and-place system.

In the sense of the invention, a “medical accessory”—or “accessory” for short—is a device, an instrument, a material, a substance or a combination thereof designated for and/or applicable to the treatment of a patient. In particular, a medical accessory can be consumed in intended use. Alternatively, a medical accessory is not consumed in intended use; i.e. is used in multiple treatments of one or more patients.

In particular, a medical accessory can be a medicinal product usually having at least substantially pharmacological, metabolic or immunological effect in intended use. Preferentially, such a medicinal product is of a pharmaceutical form, preferably for instance capsules, pills, tablets, pastes, ointments, injection doses or drug solutions of a dosage predetermined for the treatment with a dosing device, administering device and/or storage device, preferably for instance capsules or tablets in a blister pack, syringes with single or multiple doses of a injectable drug, medical syrups in bottles with or without dosage measuring cups or pharmaceutical preparations in a spray bottle having a spraying system.

In particular, a medical accessory can be also a medicinal product usually having at least substantially somatic, physical or physicochemical effect in intended use and/or by means of which a pharmaceutical is administered. Such medicinal products are in particular surgical instruments such as surgical hooks, retractors, spreaders, scalpels, scissors or forceps. Such medicinal products are in particular also needles, syringes, cannulas, Seldinger wires or catheters.

Protective gear for patients or individuals participating in the treatment can also be a medical accessory in the sense of the invention. Such protective gear in particular includes gloves to protect against chemicals or infection, preferably disposable gloves for instance made of latex, nitrile rubber or vinyl, medical mouthguards, medical hair nets or protective glasses. In particular, disinfectants, dressing materials or cosmetic products can also be medical accessories in the sense of the invention.

A medical accessory in the sense of the invention constitutes in particular a device, an instrument, a material, a substance or a combination thereof for patient cannulation, preferably for instance for a dialysis treatment. This in particular includes syringes, cannulas, infusion tubes, disposable gloves, disinfectants, swabs, dressing material, bandages, tapes and adhesive strips.

In addition, a medical accessory in the sense of the invention has physical dimensions and a weight which enables an individual participating in the treatment to transport and/or handle the medical accessory. Thus, the weight of such a medical accessory is in particular no more than 10 kg, preferably no more than 1 kg, preferably no more than 300 g, preferably no more than 100 g and further preferentially at the most 30 g. The physical dimensions to such a medical accessory are in particular of a length of no more than 1.5 m, preferably no more than 1 m, preferably no more than 0.5 m, preferably no more than 30 cm, preferably no more than 15 cm and further preferentially at the most 5 cm, wherein the same applies to the width and height. It is thereby needless to say that particularly the physical dimensions of such a medical accessory, such as an infusion tube or a tape, can also vary upon being unrolled, rolled or sectioned, whereby relevant to the handling or transport is that the physical dimensions of such a medical accessory is variable to the extent of exhibiting no more than the length, width and depth indicated above.

Preferably, the medical accessory in the sense of the invention can be designed and in particular configured such that a treatment apparatus for treating a patient can utilize the medical accessory in the treatment; in particular accommodate, operate, manipulate and/or administer.

As defined by the invention, a “medical accessory set”—or “accessory set” for short—is an assemblage of one or more medical accessories. Preferably, a medical accessory set is provided for a specific treatment and/or a specific patient and comprises thereto at least one medical accessory which is required, in particular suitable and/or selected for said patient and/or said treatment.

In the sense of the invention, a “medical accessory holder”—or “accessory holder” for short—is to be understood as at least one apparatus for supporting and/or suspending a medical accessory set. Preferably, the accessory holder can comprise one, preferably at least two further, preferably all of the medical accessories of the accessory set, thus in particular be fitted with said medical accessories. In particular, the accessory holder can be pre-equipped with at least part of an accessory set so as to already comprise at least said part of the accessory set. Doing so enables providing accessory holders with specific, in particularly frequently needed, accessory sets, in particular without their needing to be individually fitted to a respective requirement or selection. It can also be provided for such an accessory holder to be equipped with a further part of an accessory set, whereby such an accessory holder can be equipped, in particular completely, with different medical accessory sets, whereby the accessory holder is on the one hand individually equipped with the respectively specific accessories of the accessory set while, on the other hand, that part of the accessory set or group of medical accessory sets which is common to same and/or frequently needed does not need to be individually equipped to a specific requirement or selection.

In particular, a medical accessory set can comprise a receiving device designed to accommodate one, preferably at least two and further preferably all of the medical accessories of the accessory set, in particular detachably connect to same in form-fit, force-fit or materially bonded manner and/or enclose same and/or wherein the medical accessories are thus arranged in the receiving device. Preferably, an accessory holder comprises one or more retention devices for respectively receiving a medical accessory of the accessory set. In particular, medical accessories for a specific treatment can thereby be specifically arranged, preferably on the basis of their order of use in the specific treatment.

In particular, the accessory holder can be configured as a medical accessory container, wherein same or the receiving device of the accessory holder comprises or consists of a container which accommodates, thus in particular spatially encloses, one, preferably at least two, and further preferably all of the medical accessories. Such an accessory container is preferably of box-shaped design and is in particular referred to as an accessory box.

Preferably, an accessory holder is configured and in particular formed such that a treatment apparatus for the treatment for which the accessory holder is intended or an individual participating in the treatment can utilize the accessory holder, thus in particular hold, open, operate, manipulate, release and/or remove the medical accessories of the accessory set with which the accessory holder is equipped.

“Treatment of a patient” in the sense of the invention refers to at least one medical; i.e. in particular therapeutic, diagnostic or cosmetic, procedure which effects changes to the body and/or health of the patient or by means of which the state of the patient's health is determined. A treatment is in particular an administration of medicinal products, a cannulation, a blood purification procedure such as dialysis, an operation and/or an examination of the patient.

A “storage apparatus” in the sense of the invention is an apparatus for storing objects, in particular devices, instruments, materials or substances, in particular of medical accessories or accessory holders. The storage apparatus can thereby preferably accommodate the objects to be stored, or at least a part thereof; i.e. in particular spatially enclose or at least partly surround them.

Preferably the storage apparatus comprises at least one storage apparatus section with an interior area, wherein the storage apparatus section spatially and physically separates the interior area from the surrounding area of the storage apparatus in a closed state. In particular, the objects to be stored can be arranged in the interior area, and thereby stored, and the interior area spatially and physically accessible from the area surrounding the storage apparatus in an open state.

Preferably, the storage apparatus comprises at least two storage areas spatially separated from one another. This enables different objects to be stored separately from each other in the at least two storage areas and, in particular, be addressed based on the storage area. Preferably, such a storage area is designed as an interior area.

Preferably, the storage apparatus comprises a climate control device for controlling or regulating the storage conditions; i.e. in particular controlling or regulating the atmospheric humidity and/or temperature. Further storage conditions are in particular a medium in the storage apparatus, or in the interior areas of the storage apparatus respectively, preferably for instance air, an inert gas, a storage liquid or bulk material for storage—such as anti-vibration polystyrene ball—, the presence and/or absence of certain chemicals such as acids or alkalis as well as the effects of radiation such as visible light, ultraviolet light, infrared light, radioactive radiation or verberation. Preferably, the storage conditions for the interior areas of the storage apparatus can be separately controlled and/or regulated.

An “accessory database” in the sense of the invention is a database for medical accessories. In particular, same comprises a database and a program code for the administration of the database; i.e. in particular the storing, reading, retrieving, modifying and/or deleting of data, in particular the data set, of the database.

Preferably, a data processing apparatus is designed to load the program code from a data storage apparatus for the database, run same as well as load at least a part of the database from a data storage apparatus for the database, and save the database as needed after a modification. In particular, to query the accessory database, information, in particular electronically or optically coded information, can be sent to the data processing apparatus, whereupon that part of the database, or data set respectively, which corresponds to the transmitted information is output via the program code. In particular, the data sets can thereby be retrieved and thus read (or with a data-changing query, also modified), with a data field of the respective data set having a specific value or is being in a specific value range.

The accessory database, in particular the database, is designed to hold data sets on medical accessories. In particular, at least one data set in each case characterizes a medical accessory and/or its properties. Preferably, each of the medical accessories recorded in the accessory database is thereby assigned an accessory identifier and the accessory identifier stored in the data sets relating to the respective accessory as information able to be electronically retrieved and in particular queried. Also preferably, the properties of the accessories are correspondingly stored in at least one of the data sets on the respective accessory as information able to be electronically retrieved and in particular queried. Preferably, the accessory database is configured as a relational database, in particular as an SQL database.

A “separating apparatus” in the sense of the invention is an apparatus which is designed to extract an object, in particular a medical accessory, at an output location, in particular from a storage apparatus and/or from a specific position in a storage apparatus, and release it at a target location, in particular at an assembling area.

To that end, the separating apparatus preferably comprises guide means, in particular guidance devices, which guide the object from the output location to the target location; i.e. in particular at least substantially limit the object's range of motion to a course from the output location to the target location and/or effect a force on the object to be selected along a course from the output location to the target location. Such guide means or guidance devices respectively are in particular magnetic fields, electric fields, a gravitational field, a directed flow of air, ducts, tubes, belt conveyors and/or rails.

Furthermore, the separating apparatus preferably comprises control means, in particular control devices, which control the output location or the target location. By controlling the output location, a specific object can be extracted from a plurality of objects which can in particular be stored in a storage apparatus. The control of the target location in addition allows the object which is to be sorted out to be delivered to a specific destination, in particular at a specific position within an assembling area. Preferably, such a control means even guides the object which is to be sorted out in a specific direction. Also preferentially and alternatively or additionally, such a control means influence or controls a guide means such that said guide means guides the object which is to be sorted out from a specific output location and/or to a specific target location. Such control means, control devices respectively, are in particular variable magnetic fields or electric fields, a flow of air flow controllable in its strength and/or direction, switches—for instance in the case of rails—, flaps and/or actuators.

In particular, the separating apparatus can also be designed as a gripper device, wherein the guidance device comprises one or more gripping members of the gripper device and the control device comprises or consists of a positioning and/or orientation device of the gripper device—for instance telescopic rods and/or articulated joints and/or hinged arms.

To be understood by a “data processing apparatus” is an apparatus configured to process data; i.e. in particular to receive data, store received data, read out stored data, transform received and/or stored and/or read data by means of logical and/or mathematical operations, store transformed data, and/or output transformed and/or read data. Preferably, such a data processing device is programmable; i.e. a program code in particular at least partially specifies the method for processing the data and at least part of said program code is modifiable.

Preferably, the data processing apparatus is a commercially available computer. Further preferentially, the data processing apparatus comprises at least one microprocessor, a non-volatile data storage, in particular a hard disk, a read-only memory (ROM) or a drive with a data medium, as well as at least one data interface. The data processing apparatus also preferably comprises a volatile electrical data storage, in particular as main memory, preferably a semiconductor memory, in particular with integrated capacitors and/or flip-flops (bistable multivibrators) for data storage, for instance dynamic RAM or static RAM.

As defined by the invention, “configured” refers to an apparatus not only being in principle suited to fulfill a specific function—for instance only after a specific program code has been loaded; i.e. the apparatus programmed or the apparatus formed in a specific way—, but the apparatus already possesses all the means necessary in order to actually fulfill the function. Preferably, the apparatus is to that end already programmed with a program code for said function and/or already configured and/or arranged that the apparatus actually fulfills the function.

One advantage of the pick-and-place system can be in medical accessory holders being respectively fitted with medical accessories suited to the treatment to be performed and/or the patient, whereby in particular the quality and/or the safety of the treatment can be increased and/or medical accessories used in treatment-specific, patient-specific and/or efficient manner. The assembly can also be automated, whereby compared to conventional hospital logistics, personnel can be freed up and/or sources of error, such as human error, can be reduced and thus treatment safety in particular increased.

From the selection of treatment-suitable medical accessories based on the accessory database, in particular with a plurality of medical accessories, patients and/or treatments, a further advantage can be, particularly with a plurality of medical accessories, patients and/or treatments, being able to select a particularly applicable medical accessory, with the quality of the treatment thus being able to be increased. In the process, a medical accessory can for instance be selected for the same treatment to be performed on one patient which is particularly suited to said patient while a different medical accessory is selected for another patient which is particularly suited to that patient. In particular, a specific type of cannula can thereby be particularly suited for cannulation of the one patient while a different type of cannula in cannulation—and for instance subsequent dialysis—provides better treatment results for the other patient and is thus better or respectively particularly suitable for the other patient. The same applies correspondingly to different subtypes of a treatment, for instance different dialysis treatment subtypes.

Moreover, selecting from a plurality of suitable medical accessories on the basis of the accessory database enables that accessory to be selected which is to be used, and consumed as applicable, on a prioritized basis in terms of procurement costs, storage costs and/or expiration date, whereby this enables achieving an efficient use of resources; i.e. in particular of the medical accessories as available and/or to be procured.

Particularly compared to a nonspecific provision of medical accessories, the accessory set selected; i.e. in particular assembled, on the basis of the accessory database and selection parameter as well as the equipping of the medical accessory holder with medical accessories pursuant to this selection enables the providing of treatment-specific and/or patient-specific medical accessories and in particular ensuring that the required medical accessories are actually available for a specific patient and/or a specific treatment, whereby treatment safety can be increased and/or medical accessories which are not required for a treatment can be prevented from being furnished and thus unavailable at least for other treatments during this period or from needing to be disposed of, for instance upon said accessories becoming contaminated. This advantageously moreover enables patients themselves to perform simple treatment steps, for which the respectively required medical accessories thereto—and in particular only them—are provided in consolidated manner in the medical accessory holder, whereby personnel can be freed up or patient satisfaction can also be increased due to their active participation.

One advantage of fitting the medical accessory holder with the accessory set can be in the medical accessory holder consolidating the individual medical accessories into one logical unit, whereby in particular transport to a patient treatment site can be simplified/or the safety increased as individual medical accessories needed for the treatment; i.e. in particular medical accessories of the accessory set, are in each case also actually available at the treatment site during treatment. Moreover, the accessory holder being equipped with the medical accessories of the accessory set for the treatment and/or the patient simplifies the handling of the medical accessories—for instance preferably when the medical accessories are utilized by a treatment apparatus, in particular for dialysis. The hygienics can also be increased, particularly since the individual medical accessories do not need to be individually handled.

According to a first preferential embodiment, same furthermore comprises a storage apparatus for one or more accessory holders, configured to store at least the medical accessory holder, and a separating apparatus for the storage apparatus for accessory holders. The selection procedure thereby further comprises the following procedural steps. One or more accessory holder parameters characterizing the medical accessory holder to be equipped with the accessory set is/are determined in one procedural step. In a further procedural step, the separating apparatus for the storage apparatus for accessory holders is actuated at least on the basis of the accessory holder parameter. Lastly, the separating apparatus for the storage apparatus for accessory holders is designed to withdraw the medical accessory holder out of the storage apparatus for accessory holders and arrange it in the assembling area pursuant to the activation based on the accessory holder parameter.

This advantageously enables the accessory holder and preferably further accessory holders to be stored and provided for equipping within the assembling area.

A further preferential embodiment moreover comprises a support apparatus for the accessory holder which is designed to support, suspend and preferably hold the accessory holder within the assembling area.

According to a further preferential embodiment, same comprises a storage apparatus for providing fitted accessory holders, configured to store at least the medical accessory holder, and a separating apparatus for the assembling area. The selection procedure thereby further comprises the following procedural steps. One or more provisioning parameters characterizing the medical accessory holder and its bearing in the storage apparatus for providing fitted accessory holders, is/are determined in one procedural step. In a further procedural step, after being fitted, the separating apparatus for the assembling area is actuated on the basis of the accessory holder parameter. Lastly, the separating apparatus for the assembling area is designed to extract the medical accessory holder from the assembling area and deliver it to the storage apparatus for providing fitted accessory holders pursuant to the activation based on the provisioning parameter. This advantageously enables the equipping of accessory holders and thereafter storing them in the storage apparatus for providing fitted accessory holders in particular up until their being selected. In particular, accessory holders can thus be pre-fitted; i.e. in particular not just immediately before being selected, whereby in particular efficiency can be increased, peak loads prevented during assembly and/or the pick-and-place system, in particular the separating apparatus for medical accessories, utilized in a more consistent manner.

According to a further preferential embodiment, one of said storage apparatus comprises a storage compartment. The storage compartment is thereby designed to accommodate one or more medical accessories of at least one of the at least two types, the medical accessory holder before or after being equipped, one or more accessory holders or a provisioned, equipped accessory holder. Moreover, one of said separating apparatus comprises an extractor device and is designed to extract a medical accessory or an accessory holder out of the storage compartment pursuant to activation by the data processing apparatus. This advantageously enables medical accessories, particularly of one type, to be separated from the remaining medical accessories. This can facilitate the extracting of such a medical accessory. The same applies correspondingly to accessory holders, which can in particular also be configured as different types. According to one preferential variant, the storage compartment is designed as a drawer, which can facilitate replenishing.

According to a further preferential embodiment, the storage apparatus for medical accessories comprises a dosing device designed to accommodate a dosable medical accessory or the separating apparatus for medical accessories comprises a dosing device, whereby the storage apparatus for medical accessories is designed to accommodate the dosable medical accessory. The selection procedure further comprises the following procedural steps. Whether the accessory set parameters characterize the dosable medical accessory is verified in one procedural step. If this is the case, one or more dosing parameters characterizing the dosable medical accessory and the dosage needed for the accessory set is/are determined. In a further procedural step, if this is the case, the dosing device is actuated at least on the basis of the dosing parameter. The dosing device is designed to withdraw a portion of the dosable medical accessory corresponding to the required dosage pursuant to the dosing parameter-based activation. In addition, the separating apparatus for medical accessories and/or the dosing device is designed to deliver said portion of the dosable medical accessory to the assembling area for the medical accessory holder pursuant to the dosing parameter and/or accessory set parameter-based activation. According to one preferential variant, a portion is separated out from the dosable medical accessory for extraction. This advantageously enables the respectively required portion to be in each case extracted from a dosable medical accessory. In particular, doing so can avoid the need to store a plurality of different dosages of one type of medical accessory. An adhesive strip, for instance, can thus be sectioned off from an adhesive strip roll according to specific need without needing to store multiple adhesive strip sections of widely varying lengths.

According to a further preferential embodiment, one of said separating apparatus comprises or consists of a gripper device and a detection device for medical accessories and/or accessory holders. The detection device is thereby configured to collect information relating to the medical accessories and/or accessory holders stored at a specific position by way of one of said storage apparatus or arranged in an area from which the separating apparatus extracts medical accessories and/or accessory holders. In addition, the selection procedure further comprises the following procedural steps. In one procedural step, the detection device collects information relating to medical accessories and/or accessory holders at the specific position. Whether a medical accessory or accessory holder arranged a specific position is characterized by the parameters of the selection process is verified in a further procedural step. If this is the case, the gripper device is actuated in a further procedural step such that the gripper device grasps said medical accessory/accessory holder for extraction and thus for selection. According to one preferential variant, if this is not the case, these procedural steps are repeated at a different position.

In the sense of the invention, a “detection device” is at least an apparatus for collecting information on objects, in particular medical accessories or accessory holders, arranged in a specific area, at a specific position or, in particular, in a specific area around a specific position. Such detection devices can in particular be imaging systems designed to capture at least one image of an object or of a specific area respectively, preferably with a camera, and in each case associate the captured image with a specific object, in particular a specific medical accessory or specific accessory holder. Such detection devices can in particular also be barcode readers or RFID readers. In particular, a detection device can be designed to determine an accessory identifier characterizing the detected medical accessory and/or an accessory holder identifier which characterizes the detected accessory holder.

One advantage of the combination of detection device and gripper device can be being able to determine the position of a specific medical accessory or a specific accessory holder and being able to subsequently select them. This in particular enables dynamic stock management to be realized, whereby individual medical accessories or accessory holders are adaptively identified such that they can in particular be flexibly stored at different positions subject to the required storage conditions and/or available storage space. A further advantage compared to fixed selection solely on the basis of predetermined positions can also be that medical accessories or accessory holders which are stored in the wrong place are detected and thus not erroneously selected.

According to a further preferential embodiment, same further comprises a data storage apparatus for an allocation rule for accessory holders as well as the allocation rule for accessory holders, wherein said allocation rule is configured to assign an accessory identifier, patient identifier and/or treatment identifier on the basis of allocation data stored in the data storage apparatus for said allocation rule. Moreover, the data processing apparatus is further configured to implement an allocation procedure. The allocation procedure comprises at least one of the two procedural steps of determining a patient identifier at least on the basis of the selection parameter, wherein the patient identifier characterizes the patient to be treated, or determining a treatment identifier at least on the basis of the selection parameter, wherein the treatment identifier characterizes the treatment to be performed. Additionally, the allocation procedure comprises the procedural step of determining an accessory holder identifier characterizing the accessory holder with the accessory set and storing allocation data characterizing a correlation between said accessory holder identifier and patient identifier and/or treatment identifier in the data storage apparatus for the allocation rule for accessory holders.

An “accessory holder identifier” in the sense of the invention is an identification which characterizes a specific accessory holder. In particular, said specific accessory holder can be identified by means of the accessory holder identifier. Such an identifier is information which can be processed by means of a data processing apparatus, thus in particular a signal sequence and/or its mechanical, electrical or optical representation in a data storage. A part of the information preferably characterizes the information as an accessory holder identifier—in particular with respect to further identifiers. An accessory holder identifier is in particular information on an accessory holder number and/or an accessory holder name, provided same is unique or is made unique by means of an additional identifier. Said information is preferably stored in readable format in an electronic data storage which is linked, in particular integrally, to the accessory holder. Thus, the accessory holder can in particular comprise an RFID tag in which its accessory holder identifier is stored. A representation of the number or the name of the accessory holder—preferably a barcode and/or a human-readable sequence of characters—can also be formed on the accessory holder as the accessory holder identifier; i.e. in particular imprinted, engraved and/or molded.

A “patient identifier” in the sense of the invention is an identification which characterizes a specific patient or a specific group of patients. The preceding description with respect to the accessory holder identifier also applies analogously to the patient identifier. In particular, the specific patient or the specific patient group to which a specific patient is assigned can be identified by means of the patient identifier. A patient identifier is thereby in particular information on a patient number, a patient name, provided it is unique or can be made unique by an additional identifier, a (health) insurance number, an ID number or unique number/name of the specific patient group.

A “group of patients” in the sense of the invention can in particular be either male or female patients, patients with a specific chronic illness, patients having a certain blood type, patients having a certain skin type, dialysis patients with arteriovenous fistula (AV fistula)—i.e. with a Cimino shunt—, dialysis patients with vascular graft access (AV graft)—i.e. in particular with a Scribner shunt—or dialysis patients who require a central venous catheter line.

A “treatment identifier” in the sense of the invention is an identification which characterizes a specific treatment or a specific group of treatments. The preceding description with respect to the accessory holder identifier also applies analogously to the treatment identifier. In particular, the specific treatment or specific treatment group to which a specific treatment is assigned can be identified by means of the treatment identifier. A treatment identifier is in particular information on a treatment number or a unique designation for a treatment or a group of treatments.

A “group of treatments” in the sense of the invention can either be specific operations, a specific illness therapy, initial patient examinations or dialysis treatments, which in turn can comprise subgroups, in particular hemodialysis, hemofiltration, hemodiafiltration, hemoperfusion or peritoneal dialysis treatments.

One advantage of the allocation rule can lie in being able to associate specific treatments and/or patients with accessory holders by means of their accessory holder identifier. Due to the allocation on the basis of allocation data which is stored in the data storage apparatus, the allocation rule can be adapted. In particular, an accessory holder can first be associated with a specific patient and/or specific treatment and the accessory holder then later associated with another patient or another treatment; or vice versa, a patient or treatment associated with a specific accessory holder.

In particular, the accessory holder identifier enables the respective accessory holder to be identified from just its identifier alone, whereby the logistics can be simplified, thus in particular the transport to the respective treatment site and/or the use of the respectively required and/or correct medical accessories of the accessory set with which the respective accessory holder is equipped can be simplified. Thus, a treatment apparatus, for instance, can identify the accessory holder required—i.e. in particular correct—for the respective treatment on this basis of its accessory holder identifier, accommodate same, and/or when the treatment apparatus accommodates or is equipped with an accessory holder, verify whether the accessory holder is suitable for the respective treatment and/or the respective patient on the basis of the accessory holder identifier.

A further preferential embodiment, wherein same comprises at least one separating apparatus for accessory holders, further comprises an identification apparatus. The identification apparatus is thereby configured to identify an individual involved in the treatment, a treatment apparatus or a transporting person or transport apparatus for accessory holders associated with a treatment and/or a patient, as well as determine the treatment identifier of the treatment and/or the patient identifier of the patient on the basis thereof. Furthermore, the data processing apparatus is further designed to implement a delivery procedure as part of the selection procedure after the accessory holder has been fitted. The delivery procedure comprises at least the following procedural steps. Determining in one procedural step one or more delivery parameters characterizing one or more accessory holders to be delivered which have an accessory holder identifier associated with the treatment identifier and/or the patient identifier by means of the allocation rule for accessory holders. In a further procedural step, the separating apparatus for accessory holders is actuated at least on the basis of the delivery parameter. The separating apparatus for accessory holders is thereby configured to extract the accessory holder to be delivered from one of said storage apparatus for accessory holders or from the assembling area and deliver it to an individual participating in the treatment, the treatment apparatus, the transporting person or the transport apparatus pursuant to the delivery parameter-based activation. In particular, the separating apparatus for accessory holders can comprise or consist of the separating apparatus for the assembling area or the separating apparatus for the storage apparatus for accessory holders.

As defined by the invention, an “individual involved in the treatment” is in particular to be understood as an attending person, for instance a physician, or an individual providing treatment support, for instance a nurse. In particular, the patient to be treated can himself also be an individual involved in the treatment or an attending person.

As defined by the invention, a “treatment apparatus” is to at least be understood as an apparatus which is equipped to perform and/or support one or more patient treatments. A treatment apparatus is in particular a surgical robot, a cannulation apparatus—in particular for a vascular access—, a dialysis apparatus or a disinfecting apparatus for disinfecting an area of a patient's body.

As defined by the invention, a “transporting person” is to be understood as an individual who transports at least medical accessories and/or accessory holders from a location where they are output, in particular by a pick-and-place system for equipping accessory holders, to a treatment site. In particular, an individual participating in the treatment can thereby also be a transporting person in the sense of the invention.

As defined by the invention, a “transport apparatus for accessory holders” is to be understood as an apparatus which is configured to transport at least accessory holders, particularly equipped with an accessory set, from a location where they are output, in particular by a pick-and-place system for equipping accessory holders, to a treatment site. An accessory holder transport apparatus is in particular a logistics robot or a pneumatic tube system.

A treatment site is in particular a location at which the respective treatment is to be performed and/or at which the medical accessories or an accessory holder fitted with an accessory set needs to be positioned in order to perform the respective treatment.

One advantage of delivering the accessory holder based on the identification can lie in being able to increase the safety and/or unauthorized persons not being able to gain access to accessory holders, in particular accessory holders with accessory sets. This also advantageously enables increasing the treatment safety and/or freeing up personnel. In particular, the transporting person or transport apparatus for accessory holders can in each case provide and/or select that respective accessory holder for transport which is to be transported to the treatment site by the transporting person or transport apparatus.

A further preferential embodiment further comprises a supply sensor apparatus designed to detect at least some of the plurality of medical accessories which are stored particularly by way of the storage apparatus. The data processing apparatus of this embodiment is furthermore designed to perform an inventory determination analysis comprising the procedural step of determining an actual supply of stocked medical accessories in each case relative to the at least two types and based on the medical accessories as detected.

A “supply sensor apparatus” in the sense of the invention is an apparatus which is designed to detect and/or determine whether there are and preferably the number of one or more objects, particularly medical accessories, stocked in a specific spatial area, in particular in a storage apparatus. In particular, a supply sensor apparatus can comprise a detection device to that end which is in particular designed to detect the specific spatial area and/or the objects arranged therein.

A supply sensor apparatus can also comprise one or more counter devices which detect the entry and exit of objects or a specific object to the area, in particular to a storage area of the storage apparatus, and thereby count the objects arranged in said area starting from an initial inventory. Preferably, the storage apparatus thereby comprises one or more storage areas, each provided for a respective specific object, in particular a specific medical accessory, and the supply sensor apparatus determines by means of the counter device—preferably for instance light barriers—the number of specific objects introduced into the respective storage area as well as the number of specific objects exiting; i.e. in particular extracted from the respective area.

A supply sensor apparatus can also comprise one or more weighing devices respectively designed to determine a weight within a specific storage area of the storage apparatus and/or acting on a specific part of the storage apparatus, in particular a support structure of the storage apparatus in a specific storage area. Moreover, such a supply sensor apparatus is designed to determine, based on the respectively determined weight, whether objects are arranged in the respective storage area and/or, based on the respectively determined weight as well as on a known weight of an object type for which the respective storage area is provided, calculate the number of objects of this type disposed therein.

This advantageously enables the pick-and-place system to be able to determine the actual inventory of medical accessories available in the storage apparatus. In particular, compared to manually determining the actual inventory or to determining the actual inventory using further apparatus, logistics can in this way be simplified, sources of error reduced and/or personnel freed up. In particular, whether and preferably to what extent medical accessories of a specific type of the at least two types are stocked can thereby be automatically determined.

According to a further preferential embodiment, the selection procedure further comprises the procedural step of verifying whether the medical accessories of the accessory set are available in the actual inventory, and only in this case performing the procedural step of actuating the separating apparatus for medical accessories. This advantageously enables ensuring that the separating apparatus is only activated to extract from the storage apparatus those medical accessories of the accessory set characterized by the accessory parameters which are also actually stocked. In particular, inaccurate assembly due in particular to undetected unstocked medical accessories can thus be prevented.

According to a further preferential embodiment, the selection procedure further comprises the following procedural steps. One further procedural step verifies whether the medical accessories of the accessory set are available in the actual inventory. If this is not the case, one or more alternative accessory set parameters characterizing an alternative accessory set for the treatment of the patient without the unstocked medical accessories as per the verification is/are determined in a further procedural step. Lastly, the separating apparatus for medical accessories is actuated at least on the basis of the alternative accessory set parameter. The accessory holder can thereby be advantageously equipped with an alternative accessory set when medical accessories of the previously specified accessory set are not available in stock, whereby a planned treatment can in particular also be performed in this case.

According to a further preferential embodiment, the inventory determination analysis further comprises the following procedural steps. One further procedural step verifies whether there are no medical accessories of one of the at least two types available in the actual inventory and/or whether the number of medical accessories of said type falls short of a predetermined limit threshold—preferably no more than 100 stocked medical accessories of said type, further preferentially no more than 50, further preferentially no more than ten and even further preferentially at the most three. Lastly, should this be the case, an accessory shortage signal is output in a further procedural step which identifies a shortage of medical accessories and preferably the shortage of the specific medical accessory and/or its type.

Signaling an accessory shortage enables being able to react to the accessory shortage. One advantage of signaling upon falling below a limit threshold can lie in being able to react early to an imminent accessory shortage so that the accessory shortage does not or only briefly occurs given a reaction of replenishing the medical accessory inventory. One advantage of the specific signaling relative to the shortage of a specific medical accessory and/or its type can lie in being able to selectively replenish the actual inventory, thus in particular being able to transport medical accessories of this type to the pick-and-place system and/or replenishing the actual inventory with same. This advantageously enables increasing efficiency, simplifying the logistics and/or freeing up personnel. Improved treatment quality and/or treatment safety can also be achieved since outages and/or delays due to non-equipped or incorrectly equipped accessory holders can be prevented.

A further preferential embodiment further comprises a sensor apparatus for accessory holders which is designed to detect information on an arrangement of an accessory holder in the assembling area for the accessory holder and output a signal relative to the arrangement to the data processing apparatus. The data processing apparatus of this embodiment is furthermore designed to receive said signal. In addition, the selection procedure moreover comprises the following procedural steps. The signal relative to the arrangement of the accessory holder is received in one procedural step. A further procedural step verifies whether the accessory holder is arranged in the assembling area for the accessory holder on the basis of the signal. Lastly, the selection procedure implements the procedural step of actuating the separating apparatus for medical accessories only if the accessory holder is disposed in the assembling area.

As defined by the invention, a “sensor apparatus for accessory holders” is at least to be understood as an apparatus designed to collect information relating to an arrangement of an accessory holder in a specific area, particular within an assembling area for accessory holders, and/or output a signal relative to said arrangement. Preferably, the sensor apparatus for accessory holders can be configured analogously to a supply sensor apparatus. It is also preferable for the sensor apparatus for accessory holders to comprise or consist of a pressure switch which is arranged within the specific area and activated when an accessory holder is disposed therein. In particular, the pressure switch can thus be arranged within the assembling area for accessory holders such that an accessory holder disposed therein activates the pressure switch. It is also preferable for the sensor apparatus for accessory holders to comprise or consist of a light barrier which spans a portion of the specific area such that an accessory holder arranged therein activates, thus in particular interrupts, the light barrier and triggers the signal.

This advantageously ensures that medical accessories of the accessory set are only delivered to the assembling area for the accessory holder if the accessory holder is in fact there. In particular, this enables improving equipping of medical accessory holders since it ensures that the accessory set to be delivered is delivered to the accessory holder and the latter equipped with same.

According to a further preferential embodiment, same comprises a placement sensor apparatus. Additionally, the separating apparatus for medical accessories is configured to deliver the medical accessories of the accessory set to the medical accessory holder and arrange them there in a predefined manner. The placement sensor apparatus is thereby designed to detect the medical accessories arranged in the now-equipped medical accessory holder. Lastly, should the detected medical accessories differ from the medical accessories of the accessory set, the data processing apparatus is designed to output an error signal identifying a differing assembly and/or repeat the selection procedure. One advantage of verifying the assembly can be at least being able to output an error signal in the event of error or preferably being able to re-equip a further accessory holder, whereby treatment errors or delays in treatment due to missing medical accessories can be prevented. One advantage of the predefined arrangement of the medical accessories can be their particularly efficient use, thus in particular withdrawal from the accessory holder, by a treatment apparatus or an individual involved in the treatment. Preferably, the medical accessories are thereby arranged in correspondence with the sequence of the treatment steps of the treatment.

According to a further preferential embodiment, same comprises a user interface which is configured to transmit user inputs to one of the data interfaces. The data processing apparatus is furthermore designed to determine the selection parameters via the user interface.

According to a further preferential embodiment, same comprises a network interface configured to receive data corresponding to the selection parameters from a network and transmit it to one of the data interfaces. The data processing apparatus is furthermore designed to determine the selection parameters based on the data from the network interface.

A further preferential embodiment, in particular for medical accessory boxes, comprises a closure apparatus which is designed to close after the medical accessory box has been equipped.

A second aspect of the invention relates to a medical accessory box for a pick-and-place system according to the first aspect of the invention or for a treatment apparatus, preferably a cannulation apparatus or dialysis apparatus. In particular, the medical accessory box is thereby an embodiment of a medical accessory holder. The medical accessory box according to the invention comprises a data storage device, a support structure for medical accessories, a closure element and a seal. The closure element is thereby designed to form, in combination with the seal and the support structure, a sterile closed interior area of the accessory box in a closed stated of said accessory box. Furthermore, the support structure preferably comprises one or more retention devices for medical accessories designed to hold the medical accessories arranged therein. The retention devices are furthermore preferably arranged such that the secured medical accessories are accommodated in the sterile closed interior area of the accessory box in the closed state. In one preferential variant, the data storage device is designed to store at least the accessory box identifier, the treatment identifier, the patient identifier, the accommodated medical accessories or a combination thereof in readable format.

The possible advantages as well as embodiments, further developments or variants of the first aspect of the invention already mentioned above also apply accordingly to the inventive medical accessory box. In particular, one advantage of the accessory box can be being able to transport the medical accessories to a treatment site, in particular to a treatment apparatus, in a sterile state. A further advantage can lie in the retention devices being able to hold the medical accessories, particularly also during transport, in a predefined arrangement. In addition, the data storage device allows identifying the accessory box by reading out the data and/or reading out patient-specific or treatment-specific data, whereby in particular the use of the accessory box by a treatment apparatus can be improved and/or the treatment safety increased.

The invention also expressly relates to a system for medical accessories having a pick-and-place system according to the first aspect of the invention and at least one treatment apparatus. Preferably, said system for medical accessories further comprises at least one transport device.

The possible advantages as well as embodiments, further developments or variants already mentioned above also apply accordingly to said inventive system for medical accessories

Further advantages, features and possible applications of the present invention are yielded by the following detailed description of at least one example embodiment and/or by the figures. Unless otherwise described or contextually indicated otherwise, the same reference numerals are substantially used to identify equivalent components in the embodiments.

Thereby shown, to some extent schematized:

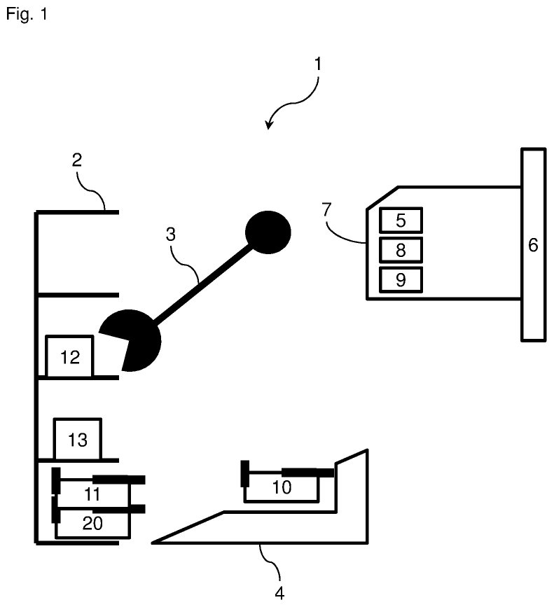

FIG. 1 one embodiment of the pick-and-place system according to the invention;

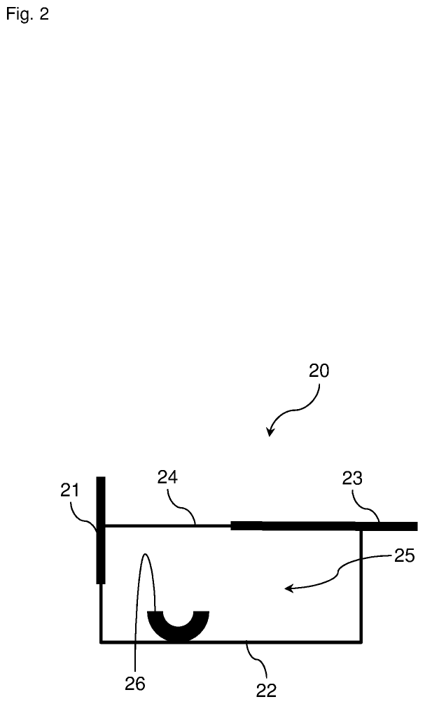

FIG. 2 one embodiment of the medical accessory box according to the invention; and

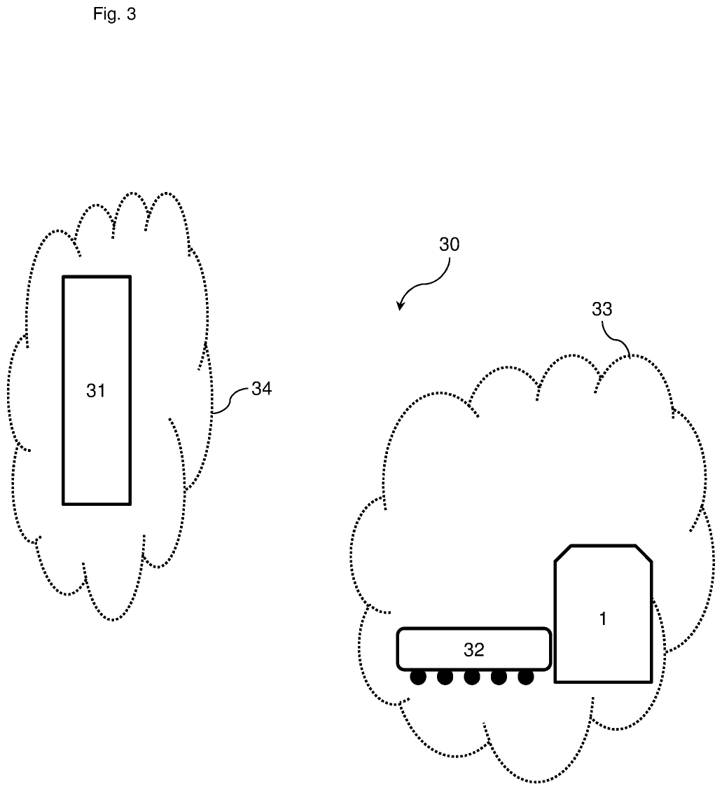

FIG. 3 one embodiment of the inventive system for medical accessories.

FIG. 1 depicts one embodiment of the pick-and-place system 1 according to the invention.

The pick-and-place system 1 comprise a storage apparatus 2 for a plurality of medical accessories as well as for one or more accessory holders. Thus, both medical accessory holders 11—which can in particular be configured as medical accessory boxes 20—as well as medical accessories 12, 13 are stored in the storage apparatus 2. The storage apparatus 2 is thereby in particular configured as a shelving system. In an alternative variant, the storage apparatus can also be designed as an arrangement of storage compartments, in particular drawers. Moreover, the pick-and-place system according to the invention can preferentially and alternatively also comprise separate storage apparatus for the medical accessories and accessory holders.

The pick-and-place system 1 comprises a separating apparatus 3 for medical accessories 12, 13 as well as for the storage apparatus 2 for accessory holders. In this embodiment, the separating apparatus 3 is configured as a gripper device. Said gripper device 3 is thereby designed to select both medical accessories 12, 13 as well as accessory holders 11, 20 from the storage apparatus 2. In selecting, a part of the gripper device is thereby moved to a specific area or a specific position respectively of the storage apparatus 2 in which a medical accessory or accessory holder to be selected is arranged—in particular, as illustrated here, to medical accessory 12 of the accessory set. The medical accessory or accessory holder is thereupon grasped by said part of the gripper device 3 and transported to an assembling area 4 for a medical accessory holder as well as released there, preferably by said part of the gripper device 3 opening. One advantage of such a combination of the storage apparatus 2 as illustrated here and the separating apparatus 3 as illustrated here can in particular lie in being able to dynamically manage the stock and there being no required predefined arrangement of the objects stored there; i.e. in particular the medical accessories as well as the accessory holder.

The pick-and-place system 1 comprises the assembling area 4 for a medical accessory holder. This is preferably designed, as illustrated, as a storage space having at least one delimitation, whereby it is in particular possible to support the accessory holder 10 as well as position it by way of the delimitation. The specific position of the accessory holder 10 can in particular facilitate the arranging of medical accessories.

The pick-and-place system 1 additionally comprises a data storage apparatus 5 for an accessory database. Said accessory database is configured to store data sets on a plurality of medical accessories and in respect of one or more patient treatments as well as enable database queries relating to the medical accessories, in particular based on a treatment to be performed or the patient to be treated. Preferably, the accessory database is configured as a relational database, in particular as an SQL database able to be queried using an SQL query.

To that end, the pick-and-place system 1 comprises a data processing apparatus 7 designed to run the accessory database. The data processing apparatus 7 moreover comprises a data interface 6 of the pick-and-place system 1, a microprocessor 8 as well as a main memory 9. Preferably, the data interface 6 is configured as a user interface. This can in particular be a touch-sensitive screen or a barcode reader or an RFID reader designed to import or respectively receive data corresponding to one or more selection parameters, particularly with treatment data or patient data, relating to the treatment of the patient. The data processing apparatus 7 is thereby designed to determine the selection parameter from said data.

The data processing apparatus 7 is moreover designed to perform a selection procedure. Said selection procedure comprises at least the following procedural steps: Determining in one procedural step one or more accessory set parameters characterizing a medical accessory set from one or more medical accessories suitable for the treatment of the patient on the basis of the accessory database and at least on the basis of the selection parameter; actuating the separating apparatus for medical accessories at least on the basis of the accessory set parameter in a further procedural step. The separating apparatus for medical accessories is thereby designed to withdraw the medical accessories of the accessory set out of the storage apparatus for medical accessories and deliver same to the assembling area for the medical accessory holder pursuant to the activation.

Lastly, the gripper device 3 is designed to move, pursuant to the activation based on the accessory set parameter, to the respective positions of the medical accessories of the accessory set, grasp the respective medical accessory there—medical accessory 12, as illustrated here—, transport same to the assembling area 4 as well as release it there. Preferably, the gripper device 3 thereby arranges the medical accessories in the medical accessory holder 10 in a predetermined manner.

FIG. 2 depicts an embodiment of the medical accessory box 20 according to the invention. The medical accessory box 20 comprises a data storage device 21, a support structure 22 for medical accessories, a closure element 23, a seal 24 as well as a retention device 26 for medical accessories. The accessory box 20 is illustrated here in an open state, whereby an interior area 25 of the accessory box 20 is indicated. This is formed when the closure element 23 is pushed toward the data storage device 21 so that the closure element 23 shuts the support structure 22 by means of the seal 24. Preferably, the accessory box 20 is designed so as to be so tightly sealed when in the closed state that the interior area 25 remains sterile.

In one preferential variant of this embodiment, the data storage device 21 comprises or consists of an RFID transmitter. In addition, the data storage device 21 is designed to store at least the accessory box identifier, the treatment identifier, the patient identifier, the accommodated medical accessories or a combination thereof in readable format.

The retention apparatus 26 for medical accessories is connected to the support structure 22, preferably in a form-fit, a material bond and/or integrally. The retention apparatus 26 is designed to receive a medical accessory in such a way as for it to be arranged at a specific position in the interior area 25 of the accessory box 20.

FIG. 3 shows a schematic representation of an embodiment of the inventive system 30 for medical accessories. Same comprises a pick-and-place system 1 preferably configured in accordance with the embodiment in respect of FIG. 1, a treatment apparatus 31 and a transport apparatus 32. The pick-and-place system 1 is thereby arranged at a first treatment site 33 and the treatment apparatus 31 arranged at a second treatment site 34.

Patients can be treated both at the first treatment site 33 as well as at a second treatment site 34. In particular, patients are thereby treated at the second treatment site 34 with treatment apparatus 31. The transport apparatus 32 thereby advantageously enables the transport of required medical accessories, in particular respectively consolidated as equipped accessory holders, from the pick-and-place system 1 at the first treatment site 33 to the treatment apparatus 31 at the second treatment site 34. This advantageously allows tending to patients at different treatment sites, which can enable flexible operation and thus efficient use of available treatment sites, and/or a respective patient having changing medical accessory needs being able to remain at one treatment site, which enables increased comfort for the patient and/or increased treatment quality.

While the preceding describes at least one preferential embodiment, it will be noted that there is a great number of variations thereof. It is also to be noted that the embodiments described only represent non-limiting examples and are not thereby intended to limit the scope, the applicability or the configuration of the apparatus and methods described herein. Rather, the foregoing description will provide a person skilled in the art with guidance for implementing at least one embodiment, wherein it is to be understood that a variety of changes can be made to the functioning and arrangement of the elements described in a preferential embodiment without thereby departing from the subject matter respectively set forth in the accompanying claims nor from legal equivalents thereof.

### LIST OF REFERENCE NUMERALS

1 pick-and-place system
2 storage apparatus for a plurality of medical accessories as well as for one or more accessory holders
3 separating apparatus for medical accessories as well as for the storage apparatus for accessory holders
4 assembling area for a medical accessory holder
5 data storage apparatus for the accessory database
6 data interface
7 data processing apparatus
8 microprocessor
9 main memory
10 medical accessory holder
11 further medical accessory holder
12 medical accessory of the accessory set
13 further medical accessory
20 medical accessory box
21 data storage device
22 support structure for medical accessories
23 closure element
24 seal
25 interior area
26 retention device for medical accessories
30 system for medical accessories
31 treatment apparatus
32 transport apparatus
33 first treatment site
34 second treatment site

1. A pick-and-place system for the equipping of medical accessory holders, for a patient treatment, wherein the pick-and-place system comprises:
at least one storage apparatus for a plurality of medical accessories of at least two types;
a separating apparatus for medical accessories;
an assembling area for a medical accessory holder;
an accessory database for data sets on the plurality of medical accessories and in respect of one or more patient treatments;
a data storage apparatus for the accessory database which is designed to store the accessory database;
one or more data interfaces, wherein at least one of the data interfaces is designed to receive one or more selection parameters relative to the treatment of the patient;
a data processing apparatus which is configured to perform a selection procedure comprising at least a procedural step of determining one or more accessory set parameters characterizing a medical accessory set from one or more medical accessories applicable to the treatment of the patient by means of the accessory database and at least on the basis of the selection parameter as well as a procedural step of actuating the separating apparatus for medical accessories at least on the basis of the accessory set parameter; and
wherein the separating apparatus for medical accessories is configured to extract the medical accessories of the accessory set from the storage apparatus for medical accessories and deliver same to the assembling area for the medical accessory holder pursuant to activation.

2. The pick-and-place system according to claim 1, comprising:
a storage apparatus for one or more accessory holders configured to store at least the medical accessory holder; and
a separating apparatus for the storage apparatus for accessory holders; and
wherein:
the selection procedure further comprises the procedural steps of determining one or more accessory holder parameters characterizing the medical accessory holder to be equipped with the accessory set and actuating the separating apparatus for the storage apparatus for accessory holders at least on the basis of the accessory holder parameter;
the separating apparatus for the storage apparatus for accessory holders is designed to withdraw the medical accessory holder out of the storage apparatus for accessory holders and arrange it in the assembling area pursuant to the activation based on the accessory holder parameter.

3. The pick-and-place system according to claim 1, comprising:
a storage apparatus for providing fitted accessory holders which is configured to store at least the medical accessory holder; and
a separating apparatus for the assembling area; and
wherein:
the selection procedure further comprises the procedural steps of determining one or more provisioning parameters characterizing the medical accessory holder and its bearing in the storage apparatus for providing fitted accessory holders and, after fitting, actuating the separating apparatus for the assembling area on the basis of the accessory holder parameter;
the separating apparatus for the assembling area is designed to withdraw the medical accessory holder from the assembling area and deliver it to the storage apparatus for providing fitted accessory holders pursuant to the activation based on the provisioning parameter.

4. The pick-and-place system according to claim 1, wherein:
one of said storage apparatus comprises a storage compartment which is designed to accommodate one or more medical accessories of at least one of the at least two types, the medical accessory holder before or after being equipped, one or more accessory holders, or a provisioned, equipped accessory holder; and
one of said separating apparatus comprises an extractor device and is designed to extract a medical accessory or an accessory holder out of the storage compartment pursuant to activation by the data processing apparatus.

5. The pick-and-place system according to claim 1, wherein:
the storage apparatus for medical accessories comprises a dosing device which is designed to accommodate a dosable medical accessory or the separating apparatus for medical accessories comprises a dosing device and the storage apparatus for medical accessories is designed to accommodate the dosable medical accessory;
the selection procedure further comprises the procedural steps of verifying whether the accessory set parameters characterize the dosable medical accessory and, should this be the case, determining one or more dosing parameters characterizing the dosable medical accessory and the dosage needed for the accessory set as well as actuating the dosing device at least on the basis of the dosing parameter;
the dosing device is designed to withdraw a portion of the dosable medical accessory corresponding to the required dosage pursuant to the dosing parameter-based activation; and
the separating apparatus for medical accessories and/or the dosing device is designed to deliver said portion of the dosable medical accessory to the assembling area for the medical accessory holder pursuant to the dosing parameter and/or accessory set parameter-based activation.

6. The pick-and-place system according to claim 1, wherein:
one of said separating apparatus comprises or consists of a gripper device and a detection device for medical accessories and/or accessory holders;
the detection device is configured to collect information relating to the medical accessories and/or accessory holders stored at a specific position by way of one of said storage apparatus or arranged in an area from which the separating apparatus extracts medical accessories and/or accessory holders;
the selection procedure further comprises the following procedural steps:
collecting information relating to medical accessories and/or accessory holders at the specific position by means of the detection device;
verifying whether a medical accessory or accessory holder arranged at the specific position is characterized by the parameters of the selection process;
if this is the case, actuating the gripper device such that said gripper device grasps said medical accessory or accessory holder respectively for extraction and thus for selection; and
if this is not the case, repeating the procedural steps at a different position.

7. The pick-and-place system according to claim 1, further comprising:
a data storage apparatus for an allocation rule for accessory holders; and
the allocation rule for accessory holders, wherein said allocation rule is configured to assign an accessory identifier, patient identifier and/or treatment identifier on the basis of allocation data stored in the data storage apparatus for said allocation rule; and
wherein the data processing apparatus is further configured to implement an allocation procedure which comprises at least one of the two procedural steps of determining a patient identifier characterizing the patient to be treated at least on the basis of the selection parameter or determining a treatment identifier characterizing the treatment to be performed at least on the basis of the selection parameter as well as further comprises at least the two procedural steps of determining an accessory holder identifier characterizing the accessory holder for or respectively with the accessory set, and storing allocation data characterizing a correlation between said accessory holder identifier and patient identifier and/or treatment identifier in the data storage apparatus for the allocation rule for accessory holders.

8. The pick-and-place system according to claim 7, comprising:
a separating apparatus for accessory holders; and
an identification apparatus which is configured to identify an individual involved in the treatment, a treatment apparatus or a transporting person or transport apparatus for accessory holders associated with a treatment and/or a patient as well as determine the treatment identifier of the treatment and/or the patient identifier of the patient on the basis thereof; and
wherein:
the data processing apparatus is further designed to implement a delivery procedure as part of the selection procedure after the accessory holder has been fitted;
the delivery procedure comprises at least the procedural steps of determining one or more delivery parameters characterizing one or more accessory holders to be delivered which have an accessory holder identifier associated with the treatment identifier and/or the patient identifier by means of the allocation rule for accessory holders as well as actuating the separating apparatus for accessory holders at least on the basis of the delivery parameter;
the separating apparatus for accessory holders is configured to extract the accessory holder to be delivered from one of said storage apparatus for accessory holders or from the assembling area and deliver it to an individual participating in the treatment, the treatment apparatus, the transporting person or the transport apparatus pursuant to the delivery parameter-based activation.

9. The pick-and-place system according to claim 1, further comprising a supply sensor apparatus designed to detect at least some of the plurality of medical accessories; and wherein the data processing apparatus is designed to perform an inventory determination analysis comprising the procedural step of determining an actual supply of stocked medical accessories in each case relative to the at least two types and based on the medical accessories as detected.

10. The pick-and-place system according to claim 9, wherein the selection procedure further comprises the procedural step of verifying whether the medical accessories of the accessory set are available in the actual inventory and only in this case performing the procedural step of actuating the separating apparatus for medical accessories.

11. The pick-and-place system according to claim 9, wherein the selection procedure further comprises the following procedural steps:
verifying whether the medical accessories of the accessory set are available in the actual inventory;
if this is not the case, determining one or more alternative accessory set parameters characterizing an alternative accessory set for the treatment of the patient without the unstocked medical accessories as per the verification; and
actuating the separating apparatus for medical accessories at least on the basis of the alternative accessory set parameter.

12. The pick-and-place system according to claim 9, wherein the inventory determination analysis further comprises the following procedural steps:
verifying whether there are no medical accessories of one of the at least two types available in the actual inventory and/or whether the number of medical accessories of said type falls short of a predetermined limit threshold; and
should this be the case, outputting an accessory shortage signal identifying a shortage of said medical accessory or its type respectively.

13. The pick-and-place system according to claim 1, further comprising a sensor apparatus for accessory holders which is designed to detect information on an arrangement of an accessory holder in the assembling area for the accessory holder and transmit a signal relative to the arrangement to the data processing apparatus; and
wherein:
the data processing apparatus is designed to receive said signal;
the selection procedure further comprises the following procedural steps:
receiving the signal relative to the arrangement of the accessory holder;
verifying whether the accessory holder is arranged in the assembling area for the accessory holder on the basis of the signal;
and the selection procedure only implementing the procedural step of actuating the separating apparatus for medical accessories if the accessory holder is disposed in the assembling area.

14. The pick-and-place system according to claim 13 comprising a placement sensor apparatus;
wherein:
the separating apparatus for medical accessories is configured to deliver the medical accessories of the accessory set to the medical accessory holder and arrange them there in a predefined manner;
the placement sensor apparatus is designed to detect the medical accessories arranged in the now-equipped medical accessory holder; and
the data processing apparatus is designed to output an error signal identifying a differing assembly should the detected medical accessories differ from the medical accessories of the accessory set and/or repeat the selection procedure.

15. A medical accessory box for a pick-and-place system according to claim 1 and/or for a treatment apparatus, wherein the medical accessory box comprises:
a data storage device;
a support structure for medical accessories;
a closure element; and
a seal;
wherein:
the closure element is designed to form, in combination with the seal and the support structure, a sterile closed interior area of the accessory box in a closed stated of said accessory box;
the support structure comprises one or more retention devices for medical accessories designed to hold the medical accessories arranged therein;
the retention devices are arranged such that the secured medical accessories are accommodated in the sterile closed interior area of the accessory box in the closed state; and
the data storage device is designed to store at least the accessory box identifier, the treatment identifier, the patient identifier, the accommodated medical accessories or a combination thereof in readable format.

16. The pick-and-place system of claim 1, wherein said medical accessory holders are medical accessory boxes.

17. The pick-and-place system of claim 1, wherein said selection parameters are treatment data or patient data.

18. The pick-and-place system of claim 4, wherein said storage component is a drawer.
